(19) United States
(12) Patent Application Publication
(10) Pub. No.: US 2008/0075745 A1
(43) Pub. Date: Mar. 27, 2008

(54) Topical pharmaceutical compositions containing ciclopirox or a pharmaceutically acceptable salt thereof

(75) Inventors: Nilendu Sen, Koperkhairne (IN); Hemalatha S. Guntur, Vashi (IN)

Correspondence Address: M. CARMEN & ASSOCIATES, PLLC, 170 OLD COUNTRY ROAD, SUITE 400, MINEOLA, NY, 11501, US

(73) Assignee: Glenmark Pharmaceuticals Limited, Andheri (East) (IN)

(21) Appl. No.: 11/903,351

(22) Filed: Sep. 21, 2007

Related U.S. Application Data
(60) Provisional application No. 60/848,828, filed on Oct. 2, 2006.

(30) Foreign Application Priority Data
Sep. 21, 2006 (IN) 1512/MUM/2006

(51) Int. Cl. A61K 9/107; A61K 31/4412; A61P 31/10

(52) U.S. Cl. 424/400; 514/350

(57) ABSTRACT

Pharmaceutical topical oil in water type emulsion gels comprising ciclopirox or a pharmaceutically acceptable salt thereof are disclosed.

### PRIORITY

This application claims the benefit under 35 U.S.C. §119 to U.S. Provisional Patent Application No. 60/848,828, filed on Oct. 2, 2006, and entitled “TOPICAL PHARMACEUTICAL COMPOSITIONS CONTAINING CICLOPIROX OR PHARMACEUTICALLY ACCEPTABLE SALTS THEREOF”, and to Indian Provisional Application 1512/MUM/2006, filed on Sep. 21, 2006, and entitled “TOPICAL PHARMACEUTICAL COMPOSITIONS CONTAINING CICLOPIROX OR PHARMACEUTICALLY ACCEPTABLE SALTS THEREOF”, the contents of each of which are incorporated by reference herein.

### BACKGROUND OF THE INVENTION

1. Technical Field

The present invention relates generally to a topically composition in emulgel form containing ciclopirox or a pharmaceutically acceptable salt thereof and a process for its preparation.

2. Description of the Related Art

There is a constant need for methods for the safe and effective administration of physiologically active agents, such as antifungal agents. For many medications, it is important that the administration regime is as simple and non-invasive as possible in order to maintain a high level of compliance by a patient. Oral administration is one administration regime that is commonly used because it is a relatively simple regime to follow. However, certain oral administration routes have been associated with complications including gastrointestinal irritation. Other adverse effects include, for example, liver damage, hepatic dysfunction, congestive heart failure, transient taste disturbance, and gastro disturbance and rashes. Poor responsiveness and relapse of the oral therapy is also common. As a number of antifungal agents are potent inhibitors of cytochrome P450 (CYP) enzymes, drug-drug interactions may affect therapeutic outcome.

Administration of physiologically active agents through the skin (i.e., topical drug delivery) has received increased attention because it not only provides a relatively simple dosage regime but it also provides a relatively slow and controlled route for release of a physiologically active agent into the local tissue. Topically administered ciclopirox (Batrafen®, Aventis Pharma Ltd, Auckland, New Zealand), amorolfine (Loceryl , Gladerma, Amersham, UK) and tioconazole (Trosyl®, Pfizer, Sandwich, UK) have demonstrated efficacy in treating nail fungal infection (onychomyosis) to some extent. Onychomycosis is known to affect the nail plate and nail bed. Topical agents capable of lateral diffusion into the infected areas would be beneficial. However, topical drug delivery is complicated by the fact that the skin behaves as a natural barrier and therefore transport of agents through the skin is a complex mechanism.

Structurally, the skin consists of two principle parts, a relatively thin outermost layer (the “epidermis”) and a thicker inner region (the ‘dermis’). The outermost layer of the epidermis (the “stratum corneum”) consists of flattened dead cells which are filled with keratin. The region between the flattened dead cells of the stratum corneum is filled with lipids which form lamellar phases that are responsible for the natural barrier properties of the skin.

For effective local delivery of a physiologically active agent that is applied to the surface of the skin (‘topical application’), the agent must be partitioned firstly from the vehicle into the stratum corneum, it must typically then be diffused within the stratum corneum before being partitioned from the stratum corneum to the local tissues including the viable epidermis, dermis, subcutis and appendageal.

To overcome some of the problems with topical delivery that are associated with transport across the dermal layers (“percutaneous absorption”), physiologically active agents are commonly formulated in such a way that they are in solubilized form in the final dosage form to allow passage through the skin barriers such that a complete therapeutic dose can be made.

Recent research has focused upon efficacious methods for development of drug delivery systems for ciclopirox or its derivatives for dermatological treatment.

U.S. Pat. No. 7,018,656 discloses a gel composition containing at least one compound chosen from 1-hydroxy-4-methyl-6-cyclohexyl-2(1H)pyridone (ciclopirox) and physiologically tolerable salts thereof; polyacrylic acid polymer; sodium dioctylsulfosuccinate; and 2-octyldodecanol.

It would be desirable to provide improved forms of topical compositions containing ciclopirox or a pharmaceutically acceptable salt thereof.

### SUMMARY OF THE INVENTION

In accordance with one embodiment of the present invention, an emulsion gel composition comprising ciclopirox or a pharmaceutically acceptable salt thereof is provided.

In accordance with a second embodiment of the present invention, a topical emulsion gel composition is provided comprising ciclopirox or a pharmaceutically acceptable salt thereof in a multiphase system.

In accordance with a third embodiment of the present invention, a topical emulsion gel composition is provided comprising ciclopirox or a pharmaceutically acceptable salt thereof wherein the gel is an oil in water emulsion gel.

In accordance with a fourth embodiment of the present invention, a process for the preparation of an emulsion gel composition is provided, the process comprising (a) dissolving a surfactant in water to form an aqueous phase; (b) preparing an oil phase; (c) adding the oil phase to the aqueous phase and homogenizing; (d) dissolving ciclopirox in a polar solvent; and (e) adding the ciclopirox solution to the oil/aqueous phase.

In accordance with a fifth embodiment of the present invention, a process for the preparation of emulsion gel composition is provided, the process comprising (a) dissolving a surfactant in water to form an aqueous phase; (b) preparing an oil phase by solubilizing cicloporox in an oil; and (c) adding the oil phase to the aqueous phase and homogenizing.

The emulsion gel composition of the present invention is advantageously in a multiphase emulsion system of ciclopirox or a pharmaceutically acceptable salt thereof and the resultant product is physico-chemically stable while providing a desired therapeutic effect.

### DETAILED DESCRIPTION OF THE INVENTION

The present invention relates to a topical emulsion gel composition containing ciclopirox or a pharmaceutically acceptable salt thereof, e.g., ciclopirox olamine, in a multiphase system such as a biphasic system. The emulsion gel described herein is a pharmaceutical composition in the form of a gel and is a multiphase system having at least an oil phase and an aqueous phase, wherein the oil phase is dispersed uniformly in the aqueous phase.

The emulsion gel composition can contain one or more solubilizers for the active ingredient, a co-solvent, gellant, an alkalizer and a vehicle. As one skilled in the art will readily appreciate, all the ingredients that are suitable to form a gel are known to one skilled in the art and can be used to prepare the emulsion gel composition of the present invention.

Solubilizers according to the present invention are a compound that is used for dissolving the therapeutically effective amount of the active ingredient. Useful solubilizers include, but are not limited to, polyethylene glycols, caffeine, xanthenes, gentisic acid, cyclodextrins, isopropyl alcohol and mixture thereof.

As will be readily be understood by those skilled in the field of pharmaceutical formulation, gels are semisolid, suspension-type systems. Gel forming agents for use herein can be any gelling agent typically used in the pharmaceutical art for topical semi solid gel dosage forms. As used herein, the term “gelling agent” is intended to mean a compound used to render a liquid vehicle into a jelly-like vehicle. Exemplary gelling agents include, by way of example and without limitation, synthetic macromolecules, cellulose derivatives (e.g. carboxymethylcellulose and hydroxypropylmethyl-cellulose) and natural gums (e.g. tragacanth). The synthetic macromolecules include carbomers (e.g. Carbomer 910, 934, 934P, 940, 941, and 1342), which are high molecular weight water-soluble polymers of acrylic acid cross-linked with allyl ethers of sucrose and/or pentaerythritol. Carbomers have different viscosities depending on their polymeric composition. Gelling agents of the present invention may be selected from any of synthetic or semi-synthetic polymeric materials, polyacrylate copolymers, cellulose derivatives and polymethyl vinyl ether/maleic anhydride copolymers. Various grades of Carbopol such as, for example, Carbopol 934, 940, 941, 974, 980, 981, 1342, 5984, ETD2020, ETD 2050, and Ultrez 10 (available from Noveon of Cleveland, Ohio) can be used in the present invention. The present invention preferably includes Carbopol 980 as a gelling agent. A Carbopol is a carbomer. Generally, carbomers are synthetic high molecular weight polymer of acrylic acid that are cross linked with either allylsucrose or allylethers of pentaerythritol.

The gelation mechanism depends on neutralization of the carboxylic acid moiety to form a soluble salt. The polymer is hydrophilic and produces sparkling clear gels when neutralized. Carbomer gels possess good thermal stability in that gel viscosity and yield value are essentially unaffected by temperature. As a topical product, carbomer gels possess optimum Theological properties. The inherent pseudo plastic flow permits immediate recovery of viscosity when shear is terminated and the high yield value and quick break make it ideal for dispensing. In the present pharmaceutical formulations, carbomer gels are used as a suspending or viscosity increasing agent. Aqueous solution of Carbopol is acidic in nature due to the presence of free carboxylic acid residues. Neutralization of this solution crosslinks and gelatinizes the polymer to form a viscous integral structure of desired viscosity. The amount of gelling agents varies widely and will ordinarily range from about 0.1% to about 10% w/w.

Suitable alkanizer agents include, but are not limited to, organic and inorganic basic compounds and the like and mixtures thereof. Representative examples of inorganic basic salts include ammonium hydroxide, alkali metal salts, alkaline earth metal salts such as magnesium oxide, magnesium hydroxide, calcium hydroxide, sodium hydroxide, potassium hydroxide, lithium hydroxide, aluminum hydroxide, potassium carbonate, sodium bicarbonate and the like and mixtures thereof.

Generally, a vehicle is a substance used to make up the volume of the composition and can be polar or non-polar solvents or a mixture thereof. Representative examples of a polar solvent include water, alcohol and the like and mixtures thereof. Solvents that are not miscible with water are non-polar solvents and include, for example, cyclohexane, carbon tetrachloride and the like. Other vehicles for use in this invention can be any other vehicle known to the person skilled in the art.

An emulsifier in a gel formulation is generally a nonionic, anionic, cationic or amphoteric surfactant. In one embodiment, a suitable emulsifier includes a nonionic surfactant such as polysorbate 60, sorbitan monostearate, polyglyceryl-4 oleate, polyoxyethylene(4)lauryl ether and the like, or trivalent cationic surfactants and sodium lauryl sulphate and mixtures thereof.

Suitable emollients for use herein include, but are not limited to, cetyl alcohol, stearyl alcohol and cetearyl alcohol, hydrocarbons, e.g., petrolatum and light mineral oil; acetylated lanolin and the like and mixtures thereof.

The oily phase of the compositions according to the invention consists of at least one oil of plant, animal, mineral or synthetic origin, which is preferably cosmetically, dermatologically or pharmaceutically acceptable. Among the plant oils which may be used include sunflower oil, corn oil, soya oil, marrow oil, grapeseed oil, blackcurrant seed oil, jojoba oil, sweet almond oil, safflower oil, sesame oil, borage oil, hazelnut oil, macadamia oil and the liquid fraction of karite butter. Plant oils which may also be used include essential oils such as an oil of eucalyptus, oil of hybrid lavender, oil of lavender, oil of vetiver, oil of Litsea cubeba, oil of lemon, oil of sandalwood, oil of rosemary, oil of camomile, oil of savory, oil of nutmeg, oil of cinnamon, oil of hyssop, oil of caraway, oil of orange, oil of geraniol, oil of prickly juniper and oil of bergamot. Among the animal oils which may use include fish oils, turtle oil, mink oil and hydrogenated squalene (or perhydrosqualene). Mineral oils which may be used include liquid paraffin and isoparaffins.

Among the synthetic oils which may be used include hydrocarbons such as isohexadecane, polydecene and polyisobutene, fatty alcohols such as octyldodecanol, isostearyl alcohol and oleyl alcohol, esters such as essential fatty acid glycerides, triglycerides of capric and caprylic acids (caprylic capric triglyceride), glyceryl monostearate, isopropyl myristate and mixtures thereof, and linear or branched fatty acid esters with fatty alcohols, such as purcellin oil (stearyl octanoate).

Other synthetic oils for use herein include silicone oils such as linear silicone oils, e.g., polydimethylsiloxane, cyclic silicone oils, e.g., cyclopentadimethylsiloxane, and derivatives thereof such as polyphenyltrimethylsiloxane and oxyethylenated or oxypropylenated polydimethylsiloxane.

Fluoro oils such as perfluorodecahydronaphthalenes, e.g., perfluorodecalin, as well as oils of polymeric fluoro oils such as perfluoropolymethyl isopropyl ethers can also be used.

According to one embodiment, a composition according to the invention incorporates at least one active substance into the oily phase and/or into the aqueous phase.

The aqueous phase of the composition of the present invention may also contain various conventional additives known to one skilled in the art. Examples of such additives include preserving agents, fragrances, pigments (e.g., TiO₂), dyestuffs, fillers, and the like.

The compositions of this invention can further contain one or more additional excipients as known to the person skilled in the art that can be used to make the ciclopirox gel composition in the form of a multiphase system.

The following examples are provided to enable one skilled in the art to practice the invention and are merely illustrative of the invention. The examples should not be read as limiting the scope of the invention as defined in the claims.

### EXAMPLE 1

Preparation of a pharmaceutical gel for topical delivery is set forth below in Table 1.

TABLE 1

| Ingredients | % w/w |
|---|---|
| Ciclopirox | about 0.77 |
| Isopropyl Alcohol | about 10-20 |
| Isopropyl myristate | about 5-10 |
| Dimethicone copolyol 190 | about 0.5-1 |
| Carbomer 980 | about 0.5-0.8 |
| Sodium lauryl sulphate | about 0.2-0.4 |
| Sodium hydroxide | q.s to adjust pH |
| Purified Water | q.s 100.0 |

Process for Its Preparation

1. Aqueous Phase

Water was heated to 60 to 65° C., and sodium lauryl sulphate was dissolved in it. Carbomer 980 was slowly dispersed in the solution under stirring to ensure complete dispersion and hydration of the carbomer while maintaining the temperature at 60 to 65° C.

2. Oil Phase

Isopropyl myristate was heated to 60 to 65° C. and maintained at this temperature.

3. Drug Solution

Ciclopirox was completely dissolved in isopropyl alcohol.

4. Emulsification

The oil phase was added to the aqueous phase (both phases maintained at 60 to 65° C.) and homogenized to achieve adequate emulsification.

5. Addition of Drug

The emulsion was stirred to 35 to 40° C. and added the above drug solution to the emulsion under stirring.

6. Cooling

The emulsion was cooled under stirring to room temperature.

7. pH Adjustment

The pH was adjusted to 6 to 7 with sodium hydroxide.

8. Addition of Dimethicone Copolyol

Dimethicone copolyol was added and stirred.

### EXAMPLE 2

Preparation of pharmaceutical gel for topical delivery is set forth below in Table 2.

TABLE 2

| Ingredients | % w/w |
|---|---|
| Ciclopirox | 0.77 |
| Isopropyl Alcohol | 10.0-20.0 |
| Caprylic capric triglyceride (CCTG) | 5.0-10.0 |
| Carbomer 980 | 0.5-0.8 |
| Sodium lauryl sulphate | 0.05-0.3 |
| Dimethicone Copolyol 190 | 0.5-1.0 |
| Sodium hydroxide | q.s to adjust pH |
| Purified Water | q.s 100.0 |

Process for Its Preparation

1. Aqueous Phase

Water was heated to 50 to 55° C. and sodium lauryl sulphate was dissolved in it. Carbomer 980 was dispersed slowly under stirring and ensured complete dispersion and hydration of carbomer while maintaining the temperature of at 50 to 55° C.

2. Drug Solution/Oil Phase

Ciclopirox was dissolved in isopropyl alcohol and CCTG mixture under stirring

3. Emulsification

The oil phase was added to the aqueous phase (maintained at 60 to 65° C.) with homogenization to achieve adequate emulsification.

4. Addition of Drug

The emulsion was stirred to 35 to 40° C. and added the above drug solution to the emulsion under stirring.

5. Addition of Dimethicone Copolyol

Dimethicone copolyol was added to the emulsion and stirred well.

6. pH Adjustment

The pH was adjusted to 6 to 7 with sodium hydroxide.

While the above description contains many specifics, these specifics should not be construed as limitations of the invention, but merely as exemplifications of preferred embodiments thereof. Those skilled in the art will envision many other embodiments within the scope and spirit of the invention as defined by the claims appended hereto.

What is claimed is:

1. A topical composition comprising ciclopirox or pharmaceutically acceptable salt thereof and one or more pharmaceutically acceptable carriers in the form of an emulsion gel.

2. The topical emulsion gel composition of claim 1 comprising ciclopirox or a pharmaceutically acceptable salt thereof in a multiphase system.

3. The topical emulsion gel composition of claim 2, comprising ciclopirox or a pharmaceutically acceptable salt thereof, wherein the multiphase system is an oil-in-water emulsion gel.

4. The topical emulsion gel composition of claim 3, wherein the oil is selected from the group consisting of isohexadecane, polydecene, polyisobutene, octyldodecanol, isostearyl alcohol, oleyl alcohol, fatty acid glycerides, triglycerides of capric and caprylic acids, glyceryl monostearate, isopropyl myristate and mixtures thereof.

5. The topical emulsion gel composition of claim 1, comprising up to about 1% w/w ciclopirox or pharmaceutically acceptable salt thereof.

6. The topical emulsion gel composition of claim 1, comprising up to about 0.77% w/w ciclopirox or pharmaceutically acceptable salt thereof.

7. The topical emulsion gel composition of claim 1, wherein the pharmaceutically acceptable carrier comprises one or more of at least one solubilizer, at least one co-solvent, at least one gelling agent, at least one alkalizer, at least one emollient, at least one emulsifier and a vehicle.

8. The topical emulsion gel composition of claim 1, wherein the ciclopirox or pharmaceutically acceptable salt thereof is in a solubilized state.

9. A process for the preparation of an emulsion gel composition, the process comprising (a) dissolving a surfactant in water to form an aqueous phase; (b) preparing an oil phase; (c) adding the oil phase to the aqueous phase and homogenizing; (d) dissolving ciclopirox in a polar solvent; and (e) adding the ciclopirox solution to the oil/aqueous phase.

10. A process for the preparation of emulsion gel composition, the process comprising (a) dissolving a surfactant in water to form an aqueous phase; (b) preparing an oil phase by solubilizing cicloporox in an oil; and (c) adding the oil phase to the aqueous phase and homogenizing.
